(19) United States
(12) Patent Application Publication
(10) Pub. No.: US 2024/0002325 A1
(43) Pub. Date: Jan. 4, 2024

(54) OXYLIPINS, PROCESSES FOR MAKING THE SAME, AND METHODS FOR USING THE SAME

(71) Applicant: THE TRUSTEES OF INDIANA UNIVERSITY, Bloomington, IN (US)

(72) Inventors: Mithun SINHA, Indianapolis, IN (US); Robert MINTO, Indianapolis, IN (US); Imran Khan MOHAMMED, Indianapolis, IN (US)

(21) Appl. No.: 18/032,733

(22) PCT Filed: Oct. 26, 2021
(86) PCT No.: PCT/US2021/056564

§ 371 Date: Apr. 19, 2023

Related U.S. Application Data
(60) Provisional application No. 63/107,626, filed on Oct. 30, 2020.

(51) Int. Cl. C07C 51/09; C07C 59/42; G01N 33/569

(52) U.S. Cl. CPC C07C 51/09; C07C 59/42; G01N 2333/31; C07B 2200/05; G01N 33/56938

(57) ABSTRACT

The present disclosure relates to oxylipins, processes for making the same, and methods of using. The process may form deuterated oxylipins that can be used as mass standards for mass spectroscopy.

### CROSS REFERENCE TO RELATED APPLICATIONS

This application claims priority to U.S. Provisional Patent Application No. 63/107,626 filed on Oct. 30, 2020, the disclosure of which is expressly incorporated herein.

### BACKGROUND

For over fifty years since silicone breast implants were first introduced in 1962, their safety has been a source of considerable controversy in the medical field, even leading to the temporary removal of implants from the U.S. market in the 1990s. Nearly 300,000 women have breast implant surgeries every year in the United States, for cosmetic augmentation, post-mastectomy breast reconstruction (breast cancer and prophylactic mastectomy) and revision of prior augmentation and reconstruction. A subset of patients with breast implants complain of a myriad of nonspecific systemic symptoms. The symptoms described include fever, myalgias, chronic fatigue, arthralgias and a host of other manifestations often associated with autoimmune illnesses. This constellation of symptoms related to implants had been named Breast Implant Illness (BII). The number of patients who opt for breast implant explantation due to complications including breast implant illness is over 30,000.

Multiple FDA-mandated studies have repeatedly found silicone gel breast implants to be safe. Of note is the fact that these symptoms have been reported in studies associated with subjects having other implants such as orthopedic implants. This implies that the underlying cause to these conditions may be associated with factors other than implant material. Therefore, it is important to decipher the underlying molecular mechanism associated with BII for a better understanding in future of all implant related illnesses in general.

Bacterial biofilms may have a possible role as a confounding factor in the pathogenesis of BII. Thus, determining what molecules could be associated with biofilm formation could provide an important clinical insight.

Bacteria in the biofilm colony interact with host lipids and leads to the formation of oxylipins. Detection of oxylipins could provide evidence of presence of bacterial biofilms and the type of bacteria present. Though some oxylipins are commercially available, many are not. This disclosure summarizes synthesis of a class of oxylipins in natural isotopic abundance (light) isotope and deuterated (heavy) isotope forms to be used as the standards for biological detection.

Though this disclosure deals with oxylipins formed due to bacterial biofilms in and around breast tissue (rich in lipids), this is applicable to biofilm infections in other adipose tissue rich regions (abdomen, hips, etc.).

### SUMMARY

Oxylipin or oxidized lipids are gaining importance for their role in bio-signaling under various pathological conditions. One compound of particular interest is an oxylipin called 10-HOME (methyl (E)-10-hydroxy-8-octadecenoate). This compound is not available commercially, and one aspect of the present disclosure is directed to a method of synthesizing 10-HOME and related and derivative compounds of 10-HOME. In accordance with one embodiment of the present disclosure two versions of 10-HOME are prepared: a deuterium-labelled (“heavy”) one and a non-isotopically enriched “normal” one. Both the versions are used in accordance with one embodiment of the present disclosure as a standard/reference for the detection of the 10-HOME in human and other living animal tissue. In one embodiment the method of detection is via liquid chromatography-mass spectrometry (LC-MS). 10-HOME can be used to create standard curves for determining instrument response to the oxylipin. In one embodiment deuterated 10-HOME is added to experimental samples as internal standards, which can be detected concurrently with the compounds in biological tissue extracts.

The compounds of the present disclosure have commercial value as they are used for detection of oxylipins in living tissues. Furthermore, the compounds are difficult to synthesize and are sensitive to acids and further oxidation. Disclosed herein is a unique method for preparing 10-HOME and related compounds.

In one aspect, the present disclosure relates to a compound of the formula I

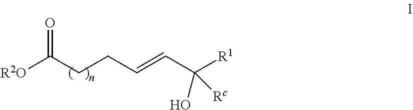

where R¹ is C₁-C₁₀ alkyl and at least one hydrogen atom in C₁-C₁₀ alkyl is substituted by deuterium,
R² is H, deuterium, C₁-C₆ alkyl, C₆-C₁₀ aryl, or heteroaryl, wherein each hydrogen atom in C₁-C₆ alkyl, C₆-C₁₀ aryl, or 3-7 membered heteroaryl is optionally substituted by deuterium,

R^{c} is H or D; and
n is an integer from 1-7;
and salts thereof. In one embodiment the compound of formula I comprises at least 3 deuterium atoms substituting for hydrogen atoms. In one embodiment the deuterium substitutions are present in the R¹ substituent.

In another aspect, the disclosure relates to processes for preparing a compound of the formula I

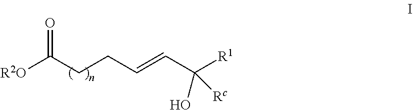

wherein R¹ is C₁-C₁₀ alkyl, further wherein each hydrogen atom in C₁-C₁₀ alkyl is optionally substituted by deuterium
R² is H, deuterium, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, heterocycloalkyl, cycloalkyl, or heteroaryl is optionally substituted by deuterium;
R^{c} is H or D; and
n is an integer from 1-7.

In another aspect, the disclosure relates to methods of using a compound of the formula I

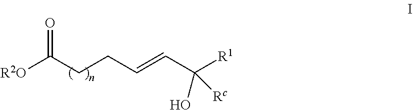

wherein R¹ is C₁-C₁₀ alkyl further wherein each hydrogen atom in C₁-C₁₀ alkyl is optionally substituted by deuterium;
R² is H, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, heterocycloalkyl, cycloalkyl, or heteroaryl is optionally substituted by deuterium;
R^{c} is H or D; and
n is an integer from 1-7.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

In one embodiment a process for forming a compound of the formula I

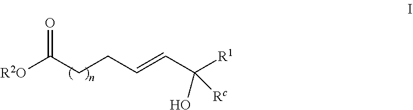

or a salt thereof is provided, wherein
   R¹ is C₁-C₁₀ alkyl, further wherein each hydrogen atom in C₁-C₁₀ alkyl is optionally substituted by deuterium;
   R² is H, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, heterocycloalkyl, cycloalkyl, or heteroaryl is optionally substituted by deuterium;
   R^{c} is H or D; and
   n is an integer from 1-7; and the process for preparing such compound comprises at least one of the following steps:
   (a) contacting a compound of formula II

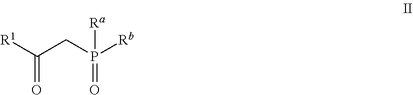

wherein R¹ is as defined above, and each of R^{a} and R^{b} is independently —OC₁-C₆ alkyl, —Oaryl, —Oheteroaryl; with a compound of formula III

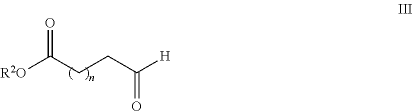

wherein R² and n are as defined above, in the presence of a base to form a compound of formula IV

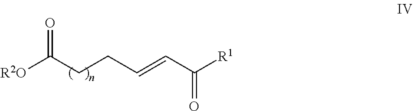

wherein R¹ and R² are as defined above; or
   (b) contacting a compound of formula IV with a reducing agent to form the compound of formula I

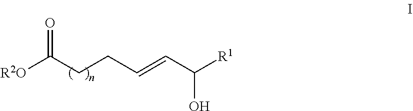

wherein R¹ and R² are as defined above; and optionally hydrolyzing the compound of formula I wherein R² is not H to form a compound of formula I wherein R² is H; or
   (c) contacting a compound of formula V

PG-O—C₁-C₉ alkyl-LG V
wherein PG is a hydroxy protecting group and LG is a leaving group, and each hydrogen atom in C₁-C₉ alkyl is optionally substituted by deuterium, with a compound of formula R³-MX, wherein R³ is C₁-C₉ alkyl, wherein each hydrogen atom in C₁-C⁹ alkyl is optionally substituted by deuterium, M is a metal, and X is a halogen, to form a compound of formula VI

PG-O—CH₂R¹ VI
wherein R¹ is as defined above and each hydrogen atom in —CH₂R¹ is optionally substituted by deuterium; or
   (d) deprotecting a compound of formula VI into a compound of formula VII

HO—CH²R¹ VII
wherein each hydrogen atom in —CH²R¹ is optionally substituted by deuterium; or
   (e) oxidizing a compound of formula VII to form a compound of formula VIII

R^{D}O(O)C—R¹ VIII
wherein R^{D} is H or C₁-C₆ alkyl; or
   (f) contacting a compound of formula VIII with (Rₐ)(R_{b})(CH₃)P(O) in the presence of a base to form a compound of formula II; or
   (g) oxidatively cleaving a C₃-C₁₀ cycloalkene with a reducing agent and optionally esterifying the product to form a compound of formula III.

In accordance with clause 2, the process of clause 1 is provided, comprising step (a).

In accordance with clause 3 the process of any one of the previous clauses is provided, comprising step (b).

In accordance with clause 4 the process of any one of the previous clauses is provided, comprising step (c).

In accordance with clause 5 the process of any one of the previous clauses is provided, comprising step (d).

In accordance with clause 6 the process of any one of the previous clauses is provided, comprising step (e).

In accordance with clause 7 the process of any one of the previous clauses is provided, comprising step (f).

In accordance with clause 8 the process of any one of the previous clauses is provided, comprising step (g).

In accordance with clause 9 the process of any one of the previous clauses is provided, comprising at least two of steps (a)-(g).

In accordance with clause 10 the process of any one of the previous clauses is provided, where said process comprises steps (a)-(b).

In accordance with clause 11 the process of any one of the previous clauses is provided, comprising steps (a), (b) and (g).

In accordance with clause 12 the process of any one of the previous clauses is provided, comprising steps (c)-(f).

In accordance with clause 13 the process of any one of the previous clauses is provided wherein
R^{c} is H; and
R¹ is C₂-C₁₀ alkyl, or C₂-C₁₀ alkyl where one or more hydrogen atoms in C₂-C₁₀ alkyl is independently, substituted by deuterium.

In accordance with clause 14 the process of any one of the previous clauses is provided wherein R¹ is C₆-C₁₀ alkyl, or C₆-C₁₀ alkyl where one or more hydrogen atoms in C₆-C₁₀ alkyl is independently, substituted by deuterium

In accordance with clause 15 the process of any one of the previous clauses is provided, wherein R¹ includes at least three deuteriums.

In accordance with clause 16 the process of any one of the previous clauses is provided, wherein the compound of formula I is

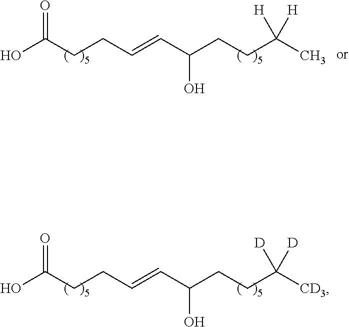

or a salt thereof.

In accordance with clause 17, a compound of formula I

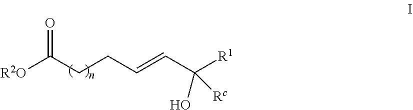

is provided wherein
R¹ is C₁-C₁₀ alkyl and at least one hydrogen atom in C₁-C₁₀ alkyl is substituted by deuterium;
R² is H, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, or 3-7 membered heteroaryl is optionally substituted by deuterium;
R^{c} is H or D; and
n is an integer from 1-7;
or a salt thereof.

In accordance with clause 18 the compound or salt of clause 17 is provided wherein R² is H.

In accordance with clause 19 the compound or salt of clause 17 or 18 is provided, wherein R^{C} is H.

In accordance with clause 20 the compound or salt of any of one clauses 17-19 is provided, wherein R¹ includes at least three deuteriums.

In accordance with clause 21. The compound or salt of any of one clauses 17-20 is provided, wherein the compound is

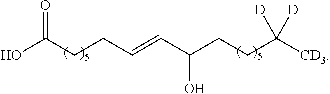

In accordance with clause 22 a method for detecting a biofilm in a patient is provided, the method comprising
collecting a sample from a patient;
adding to the sample a compound or salt thereof of any of clauses 17-21, to form a spiked sample; and
analyzing the spiked sample with mass spectrometry.

In accordance with clause 23 the method of clause 22 is provided, wherein the compound of formula I is

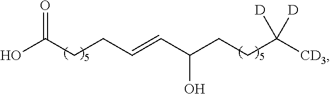

or a salt thereof.

In accordance with clause 24 the method of clause 17 or 18 is provided, wherein the sample is from a breast implant.

In accordance with clause 25 a method for detecting a biofilm in a patient is provided, wherein the method comprises
collecting a sample from a patient;
adding to the sample a compound or salt thereof made according to the process of any one of clauses 1-16, to form a spiked sample; and
analyzing the spiked sample with mass spectrometry.

In accordance with clause 26 the method of clause 25 is provided, wherein the compound of formula I is

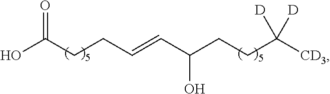

or a salt thereof.

In accordance with clause 27 the method of clause 25 or 26 is provided, wherein the sample is from a breast implant.

### BRIEF DESCRIPTION OF THE DRAWINGS

FIGS. 1A-1D show bacterial biofilm associated with breast implants. FIG. 1A provides a schematic presentation of the bacterial biofilm association with breast implant. FIG. 1B shows a breast implant isolated from a subject; and FIG. 1C shows a capsule associated with breast implant of the subject shown in FIG. 1B; FIG. 1D is a photo showing the presence of bacterial biofilm in capsules surrounding breast implant Fig.

FIGS. 2A-2I show increased abundance of biofilm-derived 10-HOME in BII subjects. FIG. 2A shows a schematic of formation of 10-HOME from oleic acid; FIGS. 2B-2D show increased abundance of 10-HOME in implant-associated tissue of BII subjects. Data presented as mean±SEM, N=6-8. FIG. 2C: Receiver operating characteristic (ROC) curve analysis to determine specificity and sensitivity of 10-HOME detection; FIG. 2D shows increased abundance of bacteria associated with 10-HOME detected from the implant associated tissue; FIGS. 2E-2H show gas chromatography analyses of extracts of Staphylococcus epidermidis (biofilm forming bacteria associated with human normal micro-flora) after in vitro culture. FIG. 2E: Oleic acid standard, FIG. 2F: 10-HOME standard, FIG. 2G: S. epidermidis with glucose as carbon source, FIG. 2H: S. epidermidis with oleic acid as carbon source. n=4. FIG. 21 is a bar graph of 10-HOME abundance in S. epidermidis cultured using glucose as carbon source vs culturing S. epidermidis using oleic acid as carbon source.

### DETAILED DESCRIPTION

Definitions

In describing and claiming the invention, the following terminology will be used in accordance with the definitions set forth below.

Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially available ACD/Name 2014 (ACD/Labs) or ChemBioDraw Ultra 19.0 (Perkin Elmer).

As used herein, the terms “including,” “containing,” and “comprising” are used in their open, non-limiting sense.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes alleviation of the symptoms associated with a specific disorder or condition and/or preventing or eliminating said symptoms.

As used herein an “effective” amount or a “therapeutically effective amount” of a drug refers to a nontoxic but enough of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein the term “patient” without further designation is intended to encompass any warm blooded vertebrate domesticated animal (including for example, but not limited to livestock, horses, cats, dogs and other pets) and humans receiving a therapeutic treatment with or without physician oversight.

The term “inhibit” defines a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 60, 70, 80, 90, 100%, or any amount of reduction in between as compared to native or control levels.

As used herein, the term “alkyl” refers to a straight or branched, saturated, aliphatic radical having the number of carbon atoms indicated. For example, C₁-C₆ alkyl includes, but is not limited to, methyl, ethyl, propyl, butyl, pentyl, hexyl, iso-propyl, iso-butyl, sec-butyl, tert-butyl, and the like. As used herein, the term “alkylene” refers to a straight or branched, saturated, aliphatic diradical having the number of carbon atoms indicated. For example, C₁-C₆ alkyl includes, but is not limited to, methylene, ethylene, propylene, butylene, pentylene, hexylene, and the like. It will be appreciated that alkyl and alkylene groups can be optionally substituted with one or more substituents by replacement of one or more hydrogen atoms on the alkyl and alkylene group.

As used herein, the term “heteroaryl” refers to a monocyclic or fused ring group of 5 to 12 ring atoms containing one, two, three or four ring heteroatoms selected from nitrogen, oxygen and sulfur, the remaining ring atoms being carbon atoms, and also having a completely conjugated pi-electron system. It will be understood that in certain embodiments, heteroaryl may be advantageously of limited size such as 3- to 7-membered heteroaryl, 5- to 7-membered heteroaryl, and the like. Heteroaryl may be unsubstituted, or substituted as described for alkyl or as described in the various embodiments provided herein. Illustrative heteroaryl groups include, but are not limited to, pyrrolyl, furanyl, thiophenyl, imidazolyl, oxazolyl, thiazolyl, pyrazolyl, pyridinyl, pyrimidinyl, quinolinyl, isoquinolinyl, purinyl, tetrazolyl, triazinyl, pyrazinyl, tetrazinyl, quinazolinyl, quinoxalinyl, thienyl, isoxazolyl, isothiazolyl, oxadiazolyl, thiadiazolyl, triazolyl, benzimidazolyl, benzoxazolyl, benzthiazolyl, benzisoxazolyl, benzisothiazolyl and carbazoloyl, and the like. Illustrative examples of heteroaryl groups shown in graphical representations, include the following entities, in the form of properly bonded moieties:

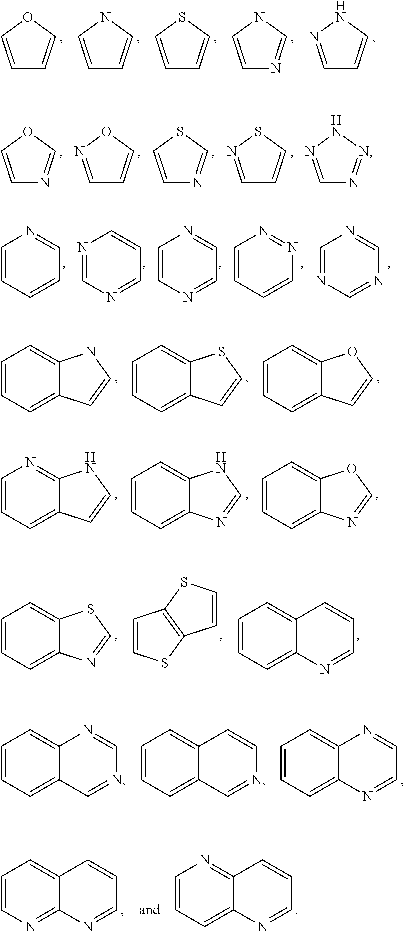

As used herein, “halogen” or “halo” refers to fluorine, chlorine, bromine, or iodine.

As used herein, “bond” refers to a covalent bond.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

As used herein, “optional” or “optionally” means that the subsequently described event or circumstance may but need not occur, and that the description includes instances where the event or circumstance occurs and instances in which it does not. For example, “wherein each hydrogen atom in C₁-C₆ alkyl is independently optionally substituted by —CN” means that a cyano may be but need not be present on the C₁-C₆ alkyl by replacing a hydrogen atom for each cyano group, and the description includes situations where the C₁-C₆ alkyl is substituted with a cyano group and situations where the C₁-C₆ alkyl is not substituted with the cyano group.

As used herein, “independently” means that the subsequently described event or circumstance is to be read on its own relative to other similar events or circumstances. For example, in a circumstance where several equivalent hydrogen groups are optionally substituted by another group described in the circumstance, the use of “independently optionally” means that each instance of a hydrogen atom on the group may be substituted by another group, where the groups replacing each of the hydrogen atoms may be the same or different. Or for example, where multiple groups exist all of which can be selected from a set of possibilities, the use of “independently” means that each of the groups can be selected from the set of possibilities separate from any other group, and the groups selected in the circumstance may be the same or different.

As used herein, the term “pharmaceutically acceptable salt” refers to those salts with counter ions which may be used in pharmaceuticals. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt. Such salts include:
(1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D)- or (L)-malic acid, maleic acid, methane sulfonic acid, ethanesulfonic acid, p-toluenesulfonic acid, salicylic acid, tartaric acid, citric acid, succinic acid or malonic acid and the like; or
(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like.

Acceptable salts are well known to those skilled in the art, and any such acceptable salt may be contemplated in connection with the embodiments described herein. Examples of acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable acceptable salts are found in Remington's Pharmaceutical Sciences, 17th Edition, Mack Publishing Company, Easton, Pa., 1985.

Any formula given herein is also intended to represent unlabeled forms as well as isotopically labeled forms of the compounds. Certain specific isotopic embodiments are also described herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C , ¹⁵N , ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements, and also for example for use as a standard in mass spectrometry. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

A biofilm-derived oxylipin may be in increased abundance in BII subjects. The oxylipin may lead to activation of CD4⁺ Th1 cells in an in vitro and in vivo mouse model indicating its role in the establishment of an autoimmune response often observed to be associated with BII. Compounds described herein may be useful in identifying the presence of oxylipins in biofilm samples.

Representative Embodiments

The compounds described herein are directed to a compound of the formula I

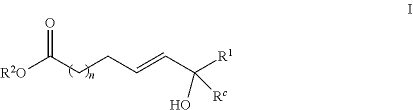

or a salt thereof.

In some embodiments, R¹ is C₁-C₁₀ alkyl. In some embodiments, each hydrogen atom in C₁-C₁₀ alkyl is optionally substituted by deuterium. In one embodiment one or more of the hydrogen atoms in the C₁-C₁₀ alkyl R¹ substituent is substituted by deuterium. In some embodiments, R¹ includes at least 2, at least 3, at least 4, at least 5, or at least 9 deuteriums In some embodiments, R¹ comprises up to about 17, up to about 14, up to about 11, or up to about 9 deuteriums. In some embodiments, R¹ is octyl. In some embodiments, R¹ is octyl and includes 8 deuteriums.

In some embodiments, R² is H, deuterium, C₁-C₆ alkyl, C₆-C₁₀ aryl, heterocycloalkyl, cycloalkyl, or heteroaryl. In one embodiment one or more of the hydrogen atoms in the C₁-C₆ alkyl, C₆-C₁₀ aryl, heterocycloalkyl, cycloalkyl, or heteroaryl R² substituent is substituted by deuterium. Illustratively, each hydrogen atom in C₁-C₆ alkyl, C₆-C₁₀ aryl, heterocycloalkyl, cycloalkyl, or heteroaryl is optionally substituted by deuterium. In some embodiments, R² is H.

In some embodiments, R^{C} is H or D. In some embodiments, R^{C} is H. In some embodiments, R^{C} is D.

In some embodiments, n is an integer selected from the range of 1-7. In some embodiments, n is 1, 2, 3, 4, 5, 6, or 7. In some embodiments, n is 5.

TABLE 1

| Compound Number | Structure | Chemical Name |
|---|---|---|
| 1 | | (E)-10-hydroxyoctadec-8-enoic acid |
| I-d₅ | | (E)-10-hydroxyoctadec-8-enoic- |
| | | 17,17,18,18,18-d₅ acid |

The compounds and process of the present disclosure are described in detail below. A step of the process of the present disclosure can be described according to Scheme 1.

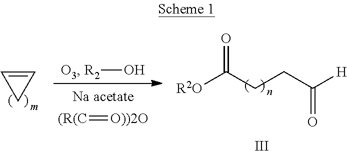

wherein n is an integer from 1 to 7, preferably 2-7, even more preferably 4 to 7. R² is H, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, heterocycloalkyl, cycloalkyl, or heteroaryl is optionally substituted by deuterium. In some embodiments, R²—OH is preferably methanol. In some embodiments, the compound of formula III can be transesterfied to form other compounds of formula III. The integer n is equal to the integer m-1. The reaction in Scheme 1 can be performed in the presence of a base such as an inorganic base. In some embodiments, the inorganic base is sodium carbonate. Illustratively, the reaction can be worked up with an acetate salt and an anhydride, preferably sodium acetate and acetic anhydride.

In some embodiments, the process of the present disclosure includes a step according to Scheme 2.

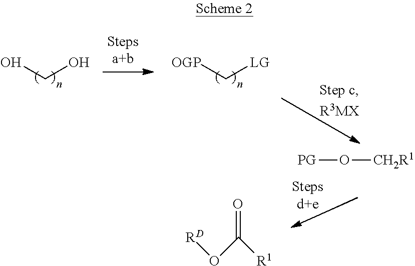

wherein n is an integer from 1-9, PG is a protecting group for a hydroxyl, LG is a leaving group, R¹ is C₁-C₁₀ alkyl and each hydrogen atom in C₁-C₁₀ alkyl is optionally substituted by deuterium, R^{D} is H or C₁-C₆ alkyl such as methyl, and R³ is C₁-C₉ alkyl, wherein each hydrogen atom in C₁-C⁹ alkyl is optionally substituted by deuterium.

Step (a) may be performed by treating the diol with a base in the presence of the protecting group precursor to protect one of the hydroxyl groups. Illustrative protecting group precursors include aryl halides, such as benzyl bromide. Step a may be performed in a solvent such as THF. The base may be sodium hydride. Illustratively, “hydroxy protecting group” means a protecting group that is protecting a hydroxy functional group.

Step (b) may be performed by treating the protected alcohol with a base in the presence of a leaving group precursor. Illustrative protecting group precursors include arylsulfonyl halides such as tosyl chloride. Step b may be performed in a solvent such as dichloromethane. The base may be an organic base, preferably triethylamine. Step (b) may also include a catalyst such as dimethylaminopyridine (DMAP).

In Step (c), the product of step (b) is contacted by R³MX. The metal, M, may be magnesium. The X may be a halide, preferably bromide. Step (c) may be performed in a solvent, preferably THF. Step (c) may be performed in the presence of a catalyst. Illustrative catalyst include copper chloride (CuCl₂). Step (c) may also be performed in the presence of a complexing ligand, such as 1-phenylpropyne.

Step (d) removes the protecting group form PG-O—CH₂R¹. In an illustrative embodiment, if the protecting group is benzyl, the benzyl is removed with boron trichloride. Alternative embodiments using different protecting groups are envisioned and the removing the protecting group is known in the art.

Step (e) oxidizes the product of step (d) to a compound of formula VIII. Step (e) may be performed using trichloroisocyanuric acid in the presence of methanol to yield a compound of formula VIII wherein R^{D} is methyl. Alternative embodiments may be performed in the presence of other alcohols. Step (e) may be performed in a solvent such as dichloromethane.

In some embodiments, the process of the present disclosure includes a step according to Scheme 3.

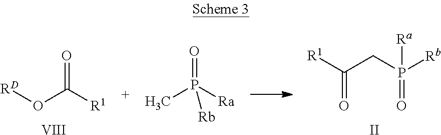

wherein R^{D} is H or C₁-C₆ alkyl such as methyl, and each of R^{a} and R^{b} is independently —OC₁-C₆ alkyl, —Oaryl, —Oheteroaryl; and R¹ is C₁-C₁₀ alkyl, wherein each hydrogen atom in C₁-C₁₀ alkyl is optionally substituted by deuterium, optionally wherein the R¹ alkyl chain substituent comprises at least 3 deuterium atoms substituting for hydrogen. In some embodiments, the phosphonate is contacted with a base prior to contacting the compound of formula VIII. Illustrative bases include alkyllithiums, such as butyllithium. In some embodiments, the reaction in scheme 3 is performed at about −78° C. In some embodiments, the compound of formula II can be purified by distillation.

In some embodiments, the process of the present disclosure includes a step according to Scheme 4.

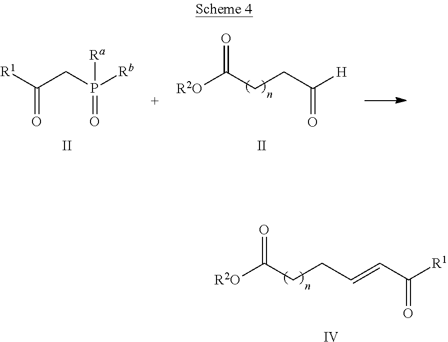

wherein R¹, Rₐ, R_{b}, R², and n are defined above. In some embodiments, the compound of formula II is contacted with a base prior to contacting the compound of formula III. Illustrative bases include sodium hydride. In some embodiments, the reaction in Scheme 4 is performed at a reduced temperature. In some embodiments, the reaction temperature increases as the reaction occurs.

In some embodiments, the process of the present disclosure includes a step according to Scheme 5.

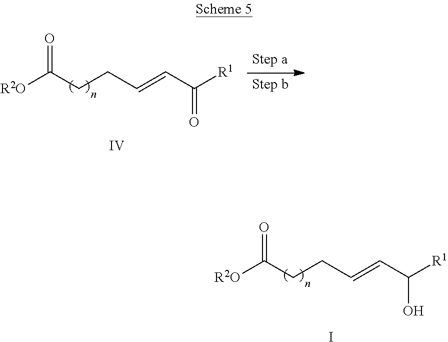

wherein R¹, R² and n are defined above. In some embodiments, Step a includes contacting the compound of formula IV with a reducing agent, preferably sodium borohydride. In some embodiments, the reducing agent delivers a deuteride instead of a hydride. In some embodiments, Step a is performed in a solvent such as a blend of an alcohol and water. In some embodiments, the reducing agent may be chiral or complexed with a chiral ligand; in some embodiments, this ligand may be the Corey-Bakshi-Shibata oxazaborolidine.

Step b is optional. If performed, Step b hydrolyzes the ester to convert R² (wherein R² is described above but is not H) to a compound of formula I wherein R² is H. In some embodiments, Step b is performed in the presence of a base in an water-organic solvent mixture. In some embodiments, the base is sodium methoxide.

In illustrative embodiments, the compound of formula I can be made by a process as described below.

In some embodiments, the process includes step (a) of contacting a compound of formula II

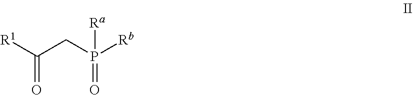

wherein R¹ is C₁-C₁₀ alkyl and each hydrogen atom in C₁-C₁₀ alkyl is optionally substituted by deuterium, and each of Rₐ and R_{b} is independently —OC₁-C₆ alkyl, —Oaryl, —Oheteroaryl; with a compound of formula III

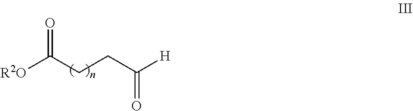

wherein R² and n are as defined above, in the presence of a base to form a compound of formula IV

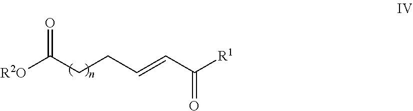

wherein R¹ and R² are as defined above.

In some embodiments, step (a) is performed in a solvent such as diethyl ether. In some embodiments, the compound of formula III is added to the compound of formula II. In some embodiments, the compound of formula II is treated with base, such as sodium hydride, prior to adding the compound of formula III. In some embodiments, the base is stoichiometric.

In some embodiments, the process includes step (b) contacting a compound of formula IV with a reducing agent to form the compound of formula I

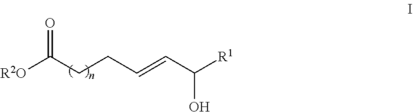

wherein R¹ and R² are as defined above, except for when R² is H. In some embodiments, the reducing agent is a borohydride, such as sodium borohydride or sodium cyanoborohydride. In some embodiments, the reducing agent provides a deuterium. In some embodiments, step (b) further includes hydrolyzing the compound of formula I, wherein R² is not H, to form a compound of formula I wherein R² is H.

In some embodiments, the process includes step (c), contacting a compound of formula V

PG-O—C₁-C₉ alkyl-LG V
wherein PG is a hydroxy protecting group and LG is a leaving group. Illustrative protecting groups include benzyl and others known in the art. Illustrative leaving groups include O-tosylates and those known in the art. Each hydrogen atom in C₁-C₉ alkyl is optionally substituted by deuterium. The compound of formula V is contacted with a compound of formula R³-MX, wherein R³ is C₁-C₉ alkyl, wherein each hydrogen atom in C₁-C⁹ alkyl is optionally substituted by deuterium, M is a metal such as magnesium, and X is a halogen such as bromo, to form a compound of formula VI

PG-O—CH₂R¹ VI

Illustratively, each hydrogen atom in —CH₂R¹ is optionally substituted by deuterium.

In some embodiments, the process includes step (d), deprotecting a compound of formula VI into a compound of formula VII

HO—CH₂R¹ VII
wherein each hydrogen atom in —CH₂R¹ is optionally substituted by deuterium. The deprotection step may depend on the nature of the protecting group. For example, if PG is a benzyl group, the deprotection may be performed by BCl₃ or any other suitable agent.

In some embodiments, the process includes step (e) oxidizing the compound of formula VII to form a compound of formula VIII

R^{D}O(O)C—R¹ VIII
wherein Ris H or C₁-C₆ alkyl such as methyl. In some embodiments, the process includes trichloroisocyanuric acid. In some embodiments, step (e) is performed in an inert atmosphere. In some embodiments, step (e) is performed in a solvent that may include more than one solvent. In illustrative embodiments, the solvent includes dichloromethane and methanol. In some embodiments, the ratio of the compound of formula VII to methanol is about 1:10.

In some embodiments, the process includes step (f), contacting a compound of formula VII with (C₁-C₆ alkoxy)₂(CH₃)P(O) in the presence of a base to form a compound of formula II. In some embodiments, the base is stoichiometric. In some embodiments, the base is n-butyllithium. In some embodiments, the (C₁-C₆ alkoxy)₂ (CH₃)P(O) is added to a flask containing the base prior to contacting the compound of formula VII.

In some embodiments, the process includes step (g), oxidatively cleaving a C₃-C10 cycloalkene and optionally esterifying the product to form a compound of formula III. In some embodiments, the oxidative cleavage is performed with ozone.

Those skilled in the art will recognize that the species listed or illustrated herein are not exhaustive, and that additional species within the scope of these defined terms may be selected.

Chemical Synthesis

Exemplary chemical entities useful in methods of the description will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups.

Glassware was oven-dried at 120° C. or flame-dried under a stream of nitrogen. Tetrahydrofuran was dried either with a commercial molecular sieves-based solvent purification system or by distillation from sodium/benzophenone. Anhydrous 1,2-dimethoxyethane was used as received. Reagents were acquired from Aldrich/Sigma Chemicals unless specified. Sodium methoxide was acquired from TCI America. NaH and phenylhydrazine were received from Merck AG and Oakwood Chemicals, respectively. Magnesium and sodium borohydride were purchased from Fisher Scientific. Butyllithium was titrated by the double Gilman titration method. The concentration of Grignard reagents was determined using salicylaldehyde phenylhydrazone. Ozone was generated from dry oxygen with an HTU-500AC unit (Azco Industries). GC-MS samples of alcohols were derivatized with N,O-bis(trimethylsilyl)trifluoroacetamide containing 2% chlorotrimethylsilane at 80° C. for 1 h. All chromatography was carried out using flash grade silica gel 60-200 mesh (60 Å pore size) from SiliCycle (Quebec City, Canada) driven by air pressure.

All proton NMR spectra were collected in CDCl₃ using Bruker Avance II 500-MHz or Avance III 400-MHz NMR spectrometers. Broadband-decoupled and DEPT ¹³C spectra were acquired in the same instruments at 125 and 100 MHz, respectively. All data was solvent referenced to δ 7.26 (¹H) and δ 77.0 ppm (¹³C). In the case of 10-HOME acid and Me ester, it was essential to deacidify CDCl₃ used for NMR samples by passage of the solvent through a plug of basic alumina. GC-MS data was collected with an Agilent 7890/5975C system using a VF-23 ms column (30 m, 250 μm film, 0.25 mm diameter) with an oven program starting at 60° C., ramp 7° C./min to 150° C., hold at 150° C. for 7 min, ramp 10° C./min to 220° C., hold 1 min, ramp 50° C./min to 250° C., and hold 2 min; He flow 1.9 mL min High-resolution mass spectra (HRMS) were collected in dual electrospray ionization (ESI) mode using an Agilent 6520 accurate mass instrument [fragmentor voltage 125V, nebulizer gas temperature 325° C., solvent: 90:10 acetonitrile:water with 0.1% formic acid].

Abbreviations The examples described herein use materials, including but not limited to, those described by the following abbreviations known to those skilled in the art:

| eq. or equiv. | equivalent |
|---|---|
| DME | 1,2-dimethoxyethane |
| TLC | thin-layer chromatography |
| min | minutes |
| h | hour |
| EtOAc | ethyl acetate |
| N | Normal |
| THF | tetrahydrofuran |
| MHz | megahertz |
| d | doublet |
| q | quintet |
| s | singlet |
| br | broad |
| NMR | nuclear magnetic resonance |
| t | triplet |
| m | multiplet |

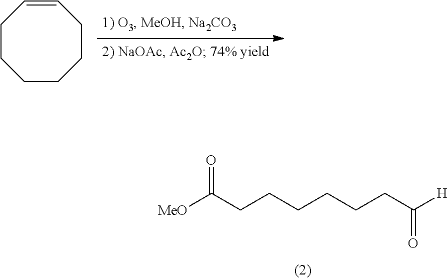 [SMILES: C1=C\CCCCCC/1 ; COC(=O)CCCCCCC=O]

Methyl 8-oxooctanoate (2): Prepared by the Schreiber variation of ozonolysis for cycloalkenes, where the ozonolysis was performed under basic conditions (NaHCO₃) in a mixture of methanol and methylene chloride, followed by treating the reaction mixture with triethylamine and acetic anhydride to produce the ester-aldehyde functionalities. The crude aldehyde was purified by distillation (53-64° C./45 mTorr) resulting in a 74% yield of 2. ¹H NMR δ 9.67 (t, J=0.9 Hz, 1H), 3.57 (s, 3H), 2.34 (m, 2H), 2.21 (m, 2H), 1.54 (m, 4H), 1.24 (m, 4H).

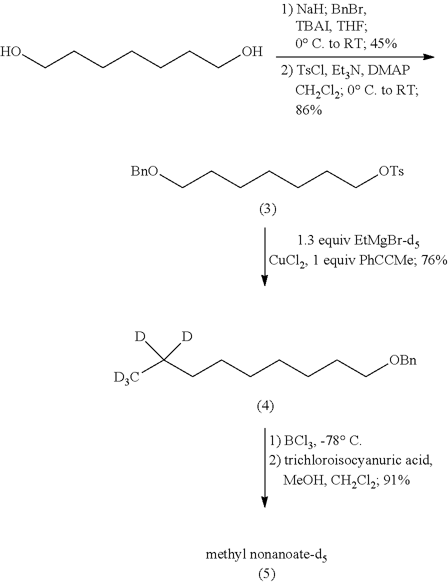 [SMILES: OCCCCCCCO ; Cc1ccc(S(=O)(=O)OCCCCCCCOCc2ccccc2)cc1 ; [2H]C([2H])([2H])C([2H])([2H])CCCCCCCOCc1ccccc1]

Methyl nonanoate (non-deuterated version of 5) was prepared in 97% yield by the esterification of nonanoic acid catalyzed by 0.005 equivalents of p-toluenesulfonic acid in the presence of methanol (10 equiv) and 2,2-dimethoxypropane (1 equiv).

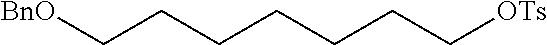 [SMILES: Cc1ccc(S(=O)(=O)OCCCCCCCOCc2ccccc2)cc1]

1-(7-Benzyloxyheptyl) tosylate (3) was prepared both by the benzylation of 1,7-heptanediol using NaH, where the intermediate benzyloxy alcohol was purified prior to tosylation, or by a KOH-based method without the intermediate purification.

Intermediate for stepwise procedure: 7-Benzyloxy-1-heptanol ¹H NMR δ 7.27-7.34 (m, 5H), 4.50 (s, 2H), 3.63 (t, J=6.6, 2H), 3.47 (t, J=7.1, 2H), 1.54-1.63 (m, 5H including br OH), 1.34-1.39 (m, 6H). (300 MHz, CDCl₃) δ 7.38-7.23 (m, 5H, ArH), 4.49 (s, 2H), 3.58 (t, J=6.5 Hz, 2H), 3.46 (t, J=6.5 Hz, 2H), 1.94 (s, 1H, OH), 1.67-1.46 (m, 5H), 1.44-1.27 (m, 6H); (75 MHz, CDCl₃) δ 138.5, 128.2, 127.5, 127.4, 72.7, 70.3, 62.7, 32.5, 29.6, 29.1, 26.0, 25.6.

1-Benzyloxy-7-tosylheptane (3): ¹H NMR δ 7.78 (d, J=7.9, 2H), 7.25-7.33 (m, 7H), 4.48 (s, 2H), 4.01 (t, J=6.4, 2H), 3.43 (t, J=6.5, 2H), 2.44 (s, 3H), 1.54-1.63 (m, 4H), 1.24-1.32 (m, 6H). HRMS (ESI) [M+H]⁺ Calc. 377.1781, Exptl. 377.1790. ¹H NMR (CDCl₃, 300 MHz) δ 7.76-7.81 (m, 2H), 7.25-7.36 (m, 7H), 4.49 (s, 2H), 4.01 (t, J=6.4, 2H), 3.44 (t, J=6.7, 2H), 2.44 (s, 3H), 1.51-1.66 (m, 4H), 1.20-1.36 (m, 6H).

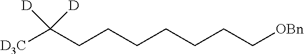 [SMILES: [2H]C([2H])([2H])C([2H])([2H])CCCCCCCOCc1ccccc1]

1-Benzyloxynonane-8,8,9,9,9-d₅ (4-d₅): In a flame-dried 50-mL Schlenk flask capped with a septum under nitrogen was placed a football stir bar, Mg turnings (360.7 mg, 15.0 mmol, 3.4 equiv), and THF (10 mL). Following the addition of 50 μL of 1,2-dibromoethane as an initiator, C₂H₅Br (750 μL, 10.0 mmol, 2.3 equiv; passed through a plug of basic alumina to remove traces of DBr) was added via a gastight syringe in portions over 26 min. The septum was replaced with a reflux condenser and the reaction was refluxed for 1 h. The concentration of the Grignard reagent measured by the phenylhydrazone method was 0.88 M. Tosylate 3 (1.6947 g, 4.38 mmol, 1.0 equiv) was placed in a dry 50-mL single-necked, round-bottomed flask containing a football-shaped magnetic stirbar. CuCl₂ (41.4 mg, 0.31 mmol, 0.7 equiv) was added, followed by 8 mL of THF, and 1-phenylpropyne (115 μL, 0.92 mmol, 0.2 equiv). The Grignard reagent was added to the tosylate-containing flask dropwise over approximately 10 minutes, resulting in a range of color changes commencing from colorless then orange brown, green, and colorless, before becoming indigo blue then finally a deep amethyst purple. The reaction was stirred for 18 h at room temperature, during which it gains a brownish tint. The reaction mixture was quenched with 1N HCl (5 mL) and saturated NH₄Cl (10 mL). The layers were separated and the aqueous layer was extracted twice with diethyl ether (10 mL each). The combined organics were washed with brine (10 mL), dried over MgSO₄, vacuum filtered through Celite and the solvent was evaporated with a rotary evaporator leading to a yellow oil (1.4018 g). The ether was purified by column chromatography (1:40 EtOAc/hexanes, 100 mL SiO₂; product R_{f} 0.32) resulting in an isolated yield of 0.8113 g of 4-d₅ (76%). ¹H NMR δ 7.29-7.44 (m, 5H), 4.54 (t, J=6.7, 2H), 1.66 (m, 2H), 1.23-1.45 (m, 10H). ¹³C NMR δ 138.71, 128.25, 127.5, 127.4, 72.79, 70.48, 31.56, 29.75, 29.54, 29.46, 29.21, 26.17, 21.6 (quint, ¹J_{CD}=19.2), 12.9 (m, ¹J_{CD}=18.6).

¹H NMR for protio coupling δ 7.27-7.35 (s, 5H), 4.50 (s, 2H), 3.47 (t, J=6.7, 2H), 1.26-1.95 (m, 14H), 0.88 (t, J=6.9, 3H). IR (neat) 1103 (C—O); ¹H NMR 7.27 (s, 5 H), 4.46 (s, 2 H), 3.43 (t, 2 H), 0.65-1.95 (m, 17 H).

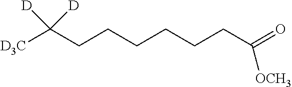 [SMILES: [2H]C([2H])([2H])C([2H])([2H])CCCCCCC(=O)OC]

Methyl nonanoate-d₅ (5-d₅):

Deprotection: In a 200-mL flask under nitrogen gas that contained benzyl ether 4-d₅ (0.760 g, 3.18 mmol, 1.0 equiv) was added a stir bar and methylene chloride (63 mL). The flask was cooled to −78° C. in a dry ice-acetone bath. BCl₃ (6.35 mL, 1M in CH₂Cl₂, 2.0 equiv) was added by syringe over 10 min, the solution changing to a light yellow-orange color. The cooling bath was swapped for an ice-water bath, and the reaction stirred for 2 h. Subsequently, the reaction mixture was re-cooled to −78° C., quenched with 2 mL of MeOH, and then allowed to warm to 0° C. in an ice-water bath. The mixture was washed with 10 mL of water and the aqueous layer was then extracted with CH₂Cl₂ (10 mL). The combined organic layers were dried over MgSO₄, vacuum filtered through Celite, and concentrated with a rotary evaporator. The initial yellow oil, which spontaneously turns green, was rapidly loaded onto a flash column (1:3 EtOAc/hexanes, 40 mL SiO₂, 1.5-cm column diameter; product R_{f} 0.49) then re-chromatographed with 1:5 EtOAc/hexanes producing 1-nonanol-d₅ (389.9 mg, 82% yield) as a colorless oil. ¹³C NMR δ 63.11, 32.82, 31.57, 29.57, 29.42, 29.21, 25.73, deuterated carbons not observed. Literature data for 1-nonanol: ¹H NMR (CDCl₃, 300 MHz) δ 3.59 (m, 2H), 2.40 (s, 1H), 1.52-1.58 (m, 2H), 1.18-1.30 (m, 12H), 0.89 (m, 3H); ¹³C NMR (CDCl₃, 75 MHz) δ 62.89, 32.70, 31.97, 29.70, 29.54, 29.39, 25.84, 22.73, 14.13.

In a 25-mL flask containing the deuterated nonanol (384.8 mg, 2.59 mmol, 1.0 equiv) and a stir bar was added CH₂Cl₂, MeOH (1.1 mL, 10 equiv). Solid trichloroisocyanuric acid (716.5 mg, 3.02 mmol, 1.17 equiv) was added and the reaction was allowed to stir for 17 h under a static N₂ atmosphere. The reaction was diluted with CH₂Cl₂ (15 mL) and vacuum filtered through Celite. The clear reaction mixture was washed twice with saturated sodium sulfite (5 mL), after which it was oxidant negative with a KI-starch test, 1N NaOH (5 mL), and saturated brine (5 mL). The methylene chloride solution was dried over MgSO₄, vacuum filtered and carefully concentrated with a room temperature bath using a rotary evaporator to a colorless oil (421.0 mg, 91% yield). ¹H NMR (CDCl₃, 300 MHz) δ 3.66 (s, 3H), 2.30 (t, J=7.6, 2H), 1.58-1.62 (m, 2H), 1.23-1.32 (m, 8H). ¹³C NMR (CDCl₃, 75 MHz) 174.3, 51.4, 34.1, 31.5, 29.21, 29.15, 29.06, 25.0, deuterated carbon signals not detected.

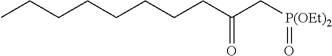

Dimethyl 2-oxodecylphosphonate (6):

In a flame-dried 1-L, 3-necked round-bottomed flask equipped with a graduated dropping funnel, septum, magnetic stir bar, and thermocouple, was placed 325 mL of tetrahydrofuran (THF) and dimethyl methylphosphonate (20.03 g, 162 mmol, 2.01 eq). The flask was cooled in a dry ice/acetone bath to an internal temperature of −70° C. n-Butyllithium (107 mL, 1.503 M in hexanes, 161 mmol, 2.0 equiv) was added dropwise via the dropping funnel over 30 min, maintaining a temperature below −60° C. and resulting in a yellow solution. After an additional 15 min of stirring, methyl nonanoate (13.866 g, 80.6 mmol, 1.0 equiv) was weighed into a transfer flask and diluted with 50 mL of THF. Over 11 minutes, the ester was added by cannula to the lithiated phosphonate solution and the reaction was stirred an additional 25 minutes. The cooling bath was replaced with a cool water bath, and the reaction mixture was allowed to warm to −0.5° C. The reaction was quenched with 100 mL of 1N HCl, the organic layer collected, and the aqueous layer was extracted with three 25-mL portions of CHCl₃. The combined organic layers were dried over MgSO₄, vacuum filtered, and concentrated with a rotary evaporator leading to 24.743 g of a crude yellow oil [containing approximately 59% w/w product]. The ketophosphonate was purified by Kuglerohr distillation at 91° C./100 mTorr, whereby side-products and excess reagents condensed in the collection bulb and the product 6 remained in the distillation pot (15.54 g, 78% yield). ¹H NMR δ 3.66 (d, J=11.2, 6H), 2.97 (d, J=22.7, 2H), 2.48 (t, J=7.3, 2H), 1.45 (m, 2H), 1.05-1.2 (m, 10H), 0.75 (t, J=6.7, 3H). ¹³C NMR δ 201.7 (d, ²J_{CP}=6.2), 52.7 (d, ²J_{CP}=6.5), 43.9, 40.9 (¹J_{CP}=128), 31.5, 29.0, 28.8, 28.6, 23.1, 22.3, 13.8.

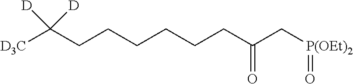

Dimethyl 2-oxodecylphosphonate-d₅ (6-d₅): An oven-dried 25-mL one-neck, round-bottomed flask containing a stirbar was cooled to −78° C. with a dry ice/acetone bath. THF (8 mL) was added by syringe followed by the slow addition of n-butyllithium (5.5 mL, 1.503 M in hexanes, 8.27 mmol, 3.5 equiv). Neat dimethyl methylphosphonate (895 μL, 8.26 mmol, 3.5 equiv) was added over 10 minutes by syringe resulting in a creamy suspension. Methyl nonanoate-d₅ (418 mg, 2.36 mmol, 1.0 equiv) dissolved in 1 mL of THF was added dropwise over 30 minutes, followed by an additional 0.3 mL of THF used to ensure complete transfer of the ester from the weighing flask. Dry ice was removed from the bath and the temperature was allowed to slowly rise to −18° C. (approx. 40 min). The reaction was quenched with 1 mL of glacial acetic acid followed by saturated NH₄Cl (10 mL). The organic layer was collected and the aqueous layer was extracted thrice with ethyl acetate (10 mL each). The combined organic layers were washed with brine (10 mL) and dried overnight with MgSO₄ Filtration and concentration with the aid of a rotary evaporator led to 1.6757 g of an oil. The crude product was purified by column chromatography (pure EtOAc, 100 mL SiO₂, 2.5-cm column diameter) providing a colorless oil 6-d₅ (571.1 mg, 90% yield). ¹H NMR δ 3.73 (d, J=11.2, 6H), 3.03 (d, J=22.7, 2H), 2.55 (t, J=7.4, 2H), 1.52 (m, 2H), 1.05-1.2 (m, 8H). ¹³C NMR δ 201.9 (d, J=6.0), 52.9 (d, J=6.4), 44.1, 41.1 (¹J_{CP}=126), 31.4, 29.2, 28.9, 28.8, 23.3, 21.4 (m), deuterated Me group not observed. HRMS (ESI) [M+H]⁺ m/z Calc. 270.1877, Exptl. 270.1887.

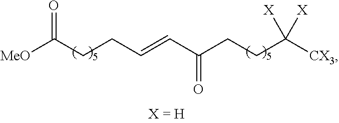

Methyl 10-oxooctadec-8-enoate (7):

In an oven-dried 25-mL 3-necked round-bottomed flask, equipped with a small stir bar and three septa was placed NaH (62.1 mg, 60% w/w in paraffin oil, 1.55 mmol, 1.1 equiv). The paraffin oil was removed by thrice washing with as-received hexanes (1-mL aliquots) then 5 mL of 1,2-dimethoxyethane (DME) was added, and the flask cooled to 0° C. in an ice-water bath. In a separate flask, a solution of phosphonate 6 (439.4 mg, 1.66 mmol, 1.2 equiv) was dissolved in 2.5 mL of DME. The phosphonate solution was added rapidly dropwise to the stirred NaH suspension by syringe, leading to a yellow solution, then the cooling bath was removed. After stirring for 1 h, neat methyl 8-oxooctanoate (240.0 mg, 1.395 mmol, 1.0 equiv) was added by syringe leading to a dense cream-colored suspension. The reaction mixture was stirred for 16 h, after which thin-layer chromatography (TLC, 1:5 v/v EtOAc/hexanes) showed that the aldehyde (R_{f} 0.29) was consumed and product had formed (R_{f} 0.53). The reaction was diluted with 10 mL of ether, and the organic phase was washed sequentially with 1N NaOH (10 mL) and brine (10 mL), then dried over MgSO₄, and concentrated by means of a rotary evaporator leading to the crude product (488.5 mg). The product was purified by flash column chromatography (1:7 EtOAc/hexanes, 50 mL SiO₂, 1.5-cm column diameter) leading to 278.2 mg of a yellowish oil (7, 57% yield). ¹H NMR δ 6.78 (dt, J=13.8, 6.9, 1H), 6.05 (dm, J=15.9, 1H), 3.63 (s, 3H), 2.48 (t, J=7.3, 2H), 2.27 (t, J=7.4, 2H), 2.17 (q, J=6.5, 2H), 1.57 (m, 4H), 1.44 (m, 2H), 1.12-1.31 (m, 16H), 0.84 (t, 3H). ¹³C NMR δ 200.8, 174.0, 146.8, 130.3, 51.3, 40.0, 33.9, 32.2, 31.7, 29.31, 29.25, 29.1, 28.8, 28.7, 27.8, 24.7, 24.3, 22.6, 14.0. GC-EIMS (22.6 min retention time, VF-23 ms column) 310, 279, 251, 212, 167 (base), 137, 119, 95, 74, 55. The (Z) isomer appeared in the lead column fraction at 20.4 min with the same fragmentation pattern. HRMS (ESI) [M+H]⁺ m/z calc. 211.2581, exptl. 211.2573.

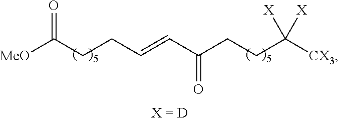

Methyl 10-oxooctadec-8-enoate—d₅ (7-d₅)

For the d₅-isotopologue, a flask was loaded with 84.6 mg of NaH (2.12 mmol, 60% w/w in oil, 1.0 equiv), and the paraffin oil removed by washing with dry hexanes before the base was suspended in 8 mL of DME. The d₅-phosphonate (6-d₅, 571.1 mg, 2.12 mmol, 1.0 equiv) was dissolved in 4-mL of DME, and added to the NaH suspension that had been cooled to 0° C. At this concentration, a creamy solid presumed to be the ylide precipitated, additional solvent was added (3 mL), and the mixture was stirred for 1 h. Neat aldehyde (428.5 mg, 1.3 equiv) was added by syringe in two portions to the flask, where mixing of the dense mixture was initially aided by manual swirling. The reaction was allowed to stir for an additional 24 h at room temperature and then diluted with 15 mL of diethyl ether. The organic phase was washed with 1N NaOH (15 mL) and then brine (15 mL) before drying with MgSO₄ and vacuum filtering the solution through a pad of Celite. Evaporation of the solvent with a rotary evaporator led to a yellow oil (904.2 mg) that was purified by flash column chromatography (1:8 EtOAc/hexanes, 80 mL SiO₂; product R_{f} 0.33) leading to 7-d₅ as a pale yellow oil (384.7 mg, 63.5% yield). ¹H NMR δ 6.80 (dt, J=6.9, 1H), 6.07 (dt, J=15.8, 1.4, 1H), 3.63 (s, 3H), 2.51 (t, J=7.3, 2H), 2.29 (t, J=7.5, 2H), 2.20 (q, J=6.5, 2H), 1.60 (m, 4H), 1.47 (m, 2H), 1.22-1.34 (m, 14H). ¹³C NMR δ 200.0, 173.4, 146.3, 130.0, 50.9, 39.6, 33.5, 31.9, 31.1, 29.0, 28.9, 28.7, 28.44, 28.38, 27.5, 24.4, 23.9, 21.2 (quint, ¹J_{CH}=18.9), 12.5 (m, ¹J_{CH}=18.8).

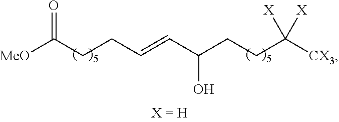

Methyl 10-hydroxyoctadec-8-enoate (10-HOME Me ester, 8):

The ketone (222 mg, 0.71 mmol, 1.0 equiv) was dissolved in 2.5 mL of methanol in a 25-mL round-bottomed flask. A slurry of NaBH₄ (54 mg, 1.42 mmol, 2.0 equiv) in 2.5-mL Me0H was transferred into the reaction flask and the mixture was magnetically stirred for 1 h at room temperature. After 1 h, additional sodium borohydride (5 mg, 0.2 equiv) was added and the mixture stirred for an additional 15 min. The solvent was removed by rotary evaporation. The residue was dissolved in 10 mL EtOAc and washed with an equal volume of brine. The organic layer was dried over MgSO₄, yielding 212.5 mg of crude product. Impurities were removed by flash column chromatography (1:7 EtOAc/hexanes, 50 mL SiO₂, 1.5-cm column) yielding 138 mg (62% yield). ¹H NMR δ 5.61 (dt, J=15.3, 6.8, 1H), 5.43 (ddt, J=15.4, 7.1, 1.3, 1H), 4.02 (q, J=6.7, 1H), 3.66 (s, 3H), 2.30 (t, J=7.5, 2H), 2.01 (q, J=6.5, 2 H), 1.23-1.64 (m, 25H), 0.87 (t, J=6.8, 3H). ¹³C NMR δ 174.3, 133.2, 131.9, 73.2, 51.4, 37.4, 34.0, 32.0, 31.9, 29.6, 29.2, 28.9, 28.7, 25.5, 24.8, 22.6, 14.1. EIMS of TMS ether m/z 384, 337, 271 (base), 241, 149, 129, 107, 73.

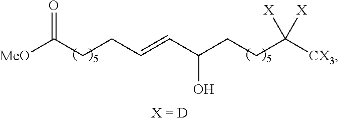

Methyl 10-hydroxyoctadec-8-enoate d₅ (10-HOME Me ester, 8-d₅):

For the d₅ -isotopologue 8-d₅, a flask was loaded with the ketoester (382 mg, 1.20 mmol, 1.0 equiv), methanol (10 mL), and a magnetic stir bar. NaBH₄ (46 mg, 1.21 mmol per addition) was added to the reaction three times, each spaced by 20-30 minutes at ambient temperature (20° C.), leading to the consumption of the ketone. The crude product (377.0 mg) was purified as for the non-deuterated compound. that was purified by flash column chromatography (1:8 EtOAc/hexanes, 80 mL SiO₂; product R_{f} 0.33) leading to a pale yellow oil (384.7 mg, 63.5% yield). ¹H NMR δ 5.61 (dt, J=15.7, 6.8, 1H), 5.43 (dd, J=15.7, 7.1, 1H), 4.02 (q, J=6.7, 1H), 3.66 (s, 3H), 2.30 (t, J=7.5, 2H), 2.01 (q, J=6.8, 2H), 1.63 (m, 4H), 1.2-1.65 (m, 19H). ¹³C NMR δ 174.2, 133.2, 131.8, 73.2, 51.4, 37.4, 34.0, 32.0, 31.6, 29.6, 29.2, 28.9, 28.7, 25.5, 24.9, deuterated carbons not observed.

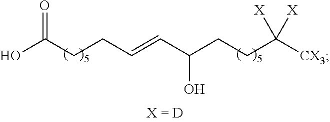

(E)-10-Hydroxyoctadec-8-enoic acid (10-HOME, 1-d₅): A 2-M solution of sodium methoxide was prepared in 15% aqueous methanol (800 μL of 5 M NaOMe in MeOH, 300-μL water, 900-μL MeOH). One milliliter of the methoxide solution was added to the deuterated 10-HOME Me ester (31.6 mg, 0.10 mmol, 1 equiv) in a glass centrifuge tube. The mixture was vortex mixed and heated for 2 h at 60° C. After cooling to room temperature, the reaction was quenched with 1 mL of 0.5 M acetic acid and 3 mL of 1 M HCl. The reaction mixture was extracted 3× with a 1:1 mixture of Et0Ac:hexanes (2 mL) and concentrated using a rotary evaporator and high vacuum pump leading to the white crystalline acid 1-d₅ (29.0 mg, 96% yield). An analogous procedure was used to hydrolyze 20.0 mg of 2 leading to white crystalline 1 (17.1 mg, 89%).

10-HOME-d₅ (1-d₅). ¹H NMR δ 6.1 (br COOH, 1H), 5.59 (dt, J=15.3, 6.7, 1H), 5.43 (dd, J=7.2, 15.3, 1H), 4.03 (q, J=6.7, 1H), 2.33 (t, J=7.5, 2H), 2.03 (q, J=6.7, 2 H), 1.62 (virt quint, J=7.1-7.4, 2H), 1.53 (m, 1H), 1.46 (m, 1H), 1.25-1.43 (m, 19H). ¹³C NMR δ 179.4, 133.0, 132.0, 73.3, 37.2, 34.0, 32.0, 31.6, 29.54, 29.52, 29.2, 28.8, 28.75, 28.6, 25.4, 24.6. HRMS (ESI) [M−H]⁻ m/z Calc. 302.2749, Exptl. 302.2734; fragment C₁₁H₁₃O₃ m/z Calc. 193.0870, Exptl. 193.0897.

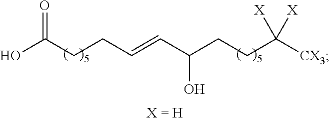

10-HOME (1) was prepared in a similar manner as above. ¹H NMR δ 5.60 (dtd, J=0.8, 15.3, 6.7, 1H), 5.44 (tdd, J=1.3, 7.1, 15.3, 1H), 4.03 (q, J=7.8, 1H), 2.34 (t, J=7.5, 2H), 2.02 (virt q, J=6.7, 2 H), 1.63 (virt quint, J=7.1-7.4, 2H), 1.47 (m, 1H), 1.38 (m, 1H), 1.26-1.43 (m, 20H), 0.87 (t, J=7.0, 3H). ¹³C NMR δ 178.9, 133.1, 132.0, 73.3, 37.3, 33.8, 32.0, 31.9, 29.5, 29.2, 28.81, 28.76, 28.6, 25.5, 24.6, 22.6, 14.1. HRMS (ESI) [M−H]⁻ m/z Calc. 297.2435, Exptl. 297.2445; fragment C₁₁H₁₃O₃ m/z Calc. 193.0870, Exptl. 193.0915.

Assays

Human subjects. Subjects participating in the study were patients diagnosed with BII. Demographic characteristics of patients presented in (Table 2). All human studies were approved by The Indiana University School of Medicine Institutional Review Board. Declaration of Helsinki protocols was followed, and patients gave their written informed consent.

Bacterial strains. Staphylococcus epidermidis (Winslow and Winslow) Evans (ATCC® 35984™) were grown on tryptic soy agar plate at 37° C.

Scanning Electron Microscope Imaging. The samples were collected in glutaraldehyde fixation buffer, dehydrated with graded ethanol, and treated with hexamethyldisilazane (HMDS, Ted Pella Inc.) and left overnight for drying. Before scanning, samples were mounted and coated with gold. Imaging of the samples will be done by using a FEI™ NOVA nanoSEM scanning electron microscope (FEI™, Hillsboro, OR) equipped with a field-emission gun electron source.

Lipid Extraction For LCMS.

LC-MS/MS targeted analysis from capsule and breast adipose tissue was performed. Samples were weighed and transferred to a 2-mL vials with 1.4-mm ceramic beads and 1 mL of water with 0.1% formic acid was added. The standard solution was prepared by aliquoting 1 μg of each stock solution into a new tube drying the original solvent and solubilizing in 1 mL of 100% ethanol to obtain a final concentration of 1 ng/ml each. Samples were homogenized using Precellys24 tissue homogenizer (Bertin Technologies, Rockville, MD, USA). The total volume of the homogenate was extracted with ethyl acetate in a 1:1 volume ratio. Samples were vortexed for 1 minute and centrifuged at 14,000 rpm for 10 minutes. The organic phase was collected and transferred to a new vial to be evaporated and stored at −80° C. until analysis. The dried lipid extracts were reconstituted with 50 μL of methanol/water at 1:1 volume ratio and submitted for targeted quantification by liquid chromatography tandem MS (LC/MS/MS). The LC column used was an Acquity UPLC BEH C18 1.7 μm 2.1×100 (Waters, Milford, MA). The binary pump flow rate was set at 0.3 mL/min in an Agilent UPLC (G7120A) using water and 0.1% formic acid as mobile phase A and acetonitrile and 0.1% formic acid as mobile phase B. The LC column was pre-equilibrated with 80% A for 1 min. The binary pump was set in a linear gradient to 100% B in 8 min and held for 2.50 min. It was then returned to 80% A and re-equilibrated for 4 min. Ten μL of the reconstituted sample was delivered to the column through a multisampler (G7167B) into a QQQ6470A triple quadrupole mass spectrometer (Agilent Technologies, San Jose, CA) equipped with ESI Jet Stream ion source. In the mass spectrometer the capillary voltage was 3500 V on the negative ion mode, the gas temperature was 325° C., gas flow was set at 8 l/min, the sheath gas heater at 250° C. and the sheath gas flow at 7. The fragmentation voltage was 100 and the cell accelerator voltage was 4 V. The MRMs (parent-fragment) for the acquisition was performed Data processing was carried out by using Mass Hunter (B.06.00).

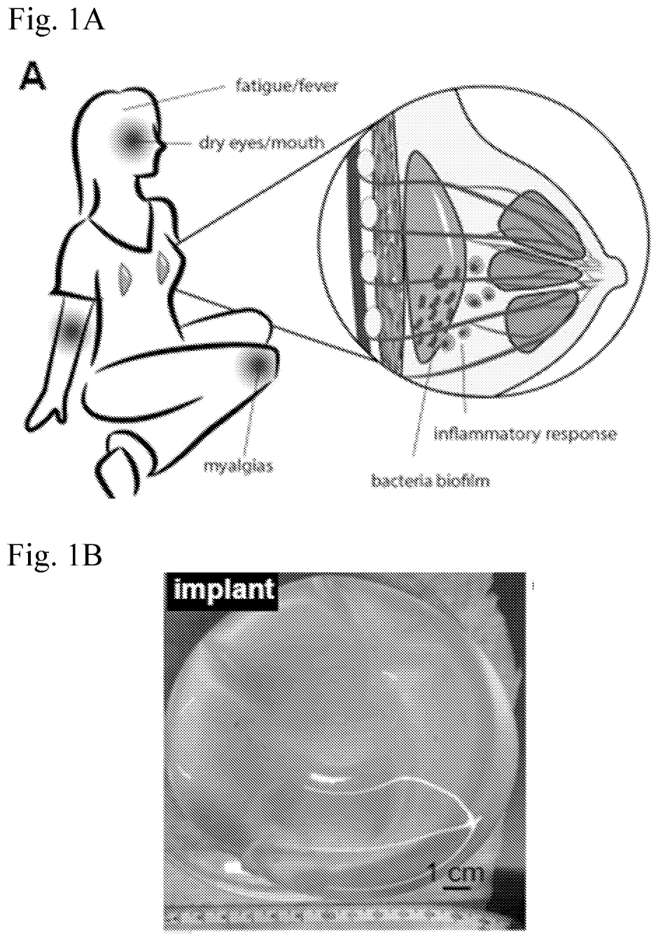

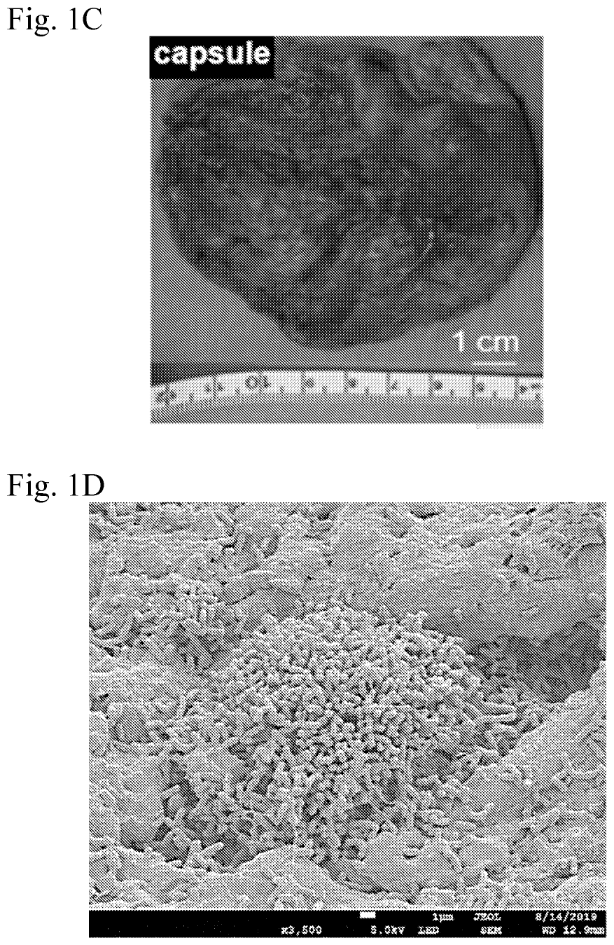

Bacterial biofilm was observed in implant-associated capsules through scanning electron microscopy (FIG. 1D).

Increased Abundance of Biofilm Derived 10-HOME in BII Subjects

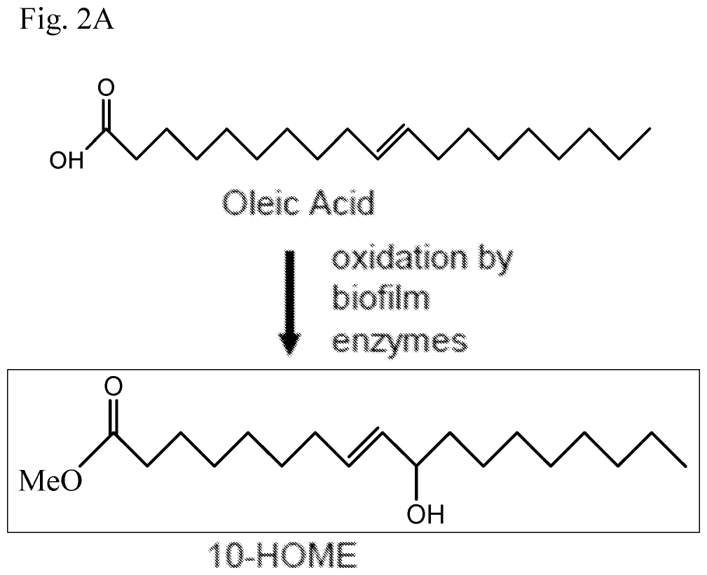

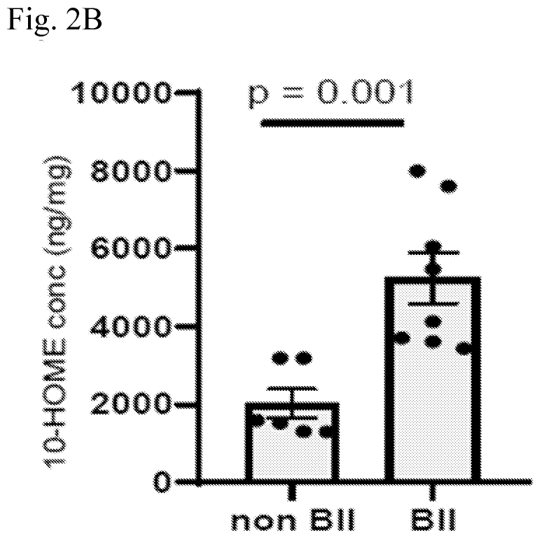

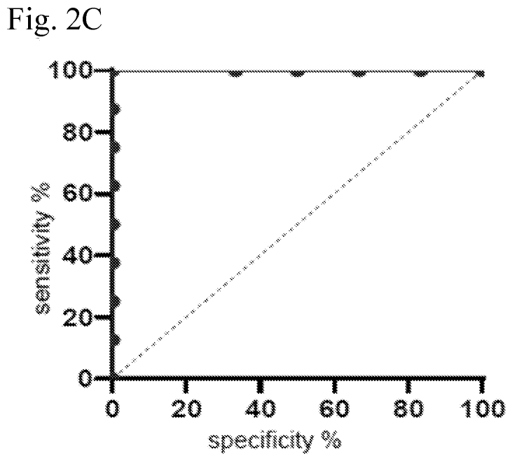

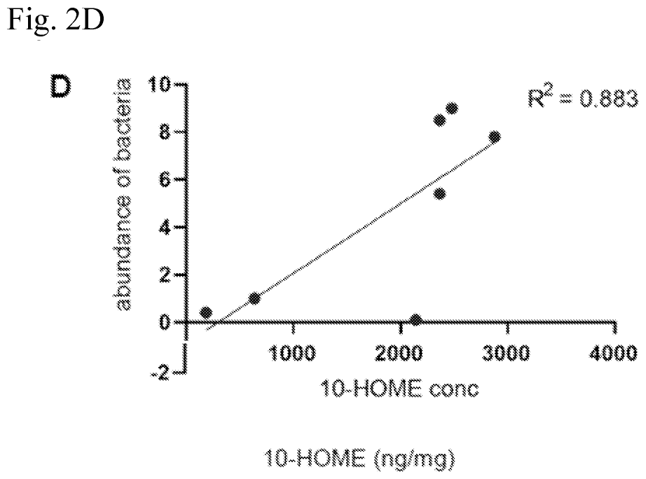

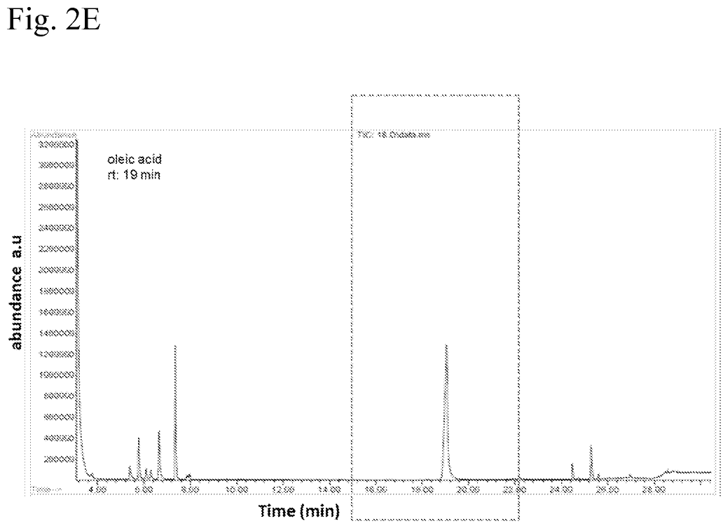

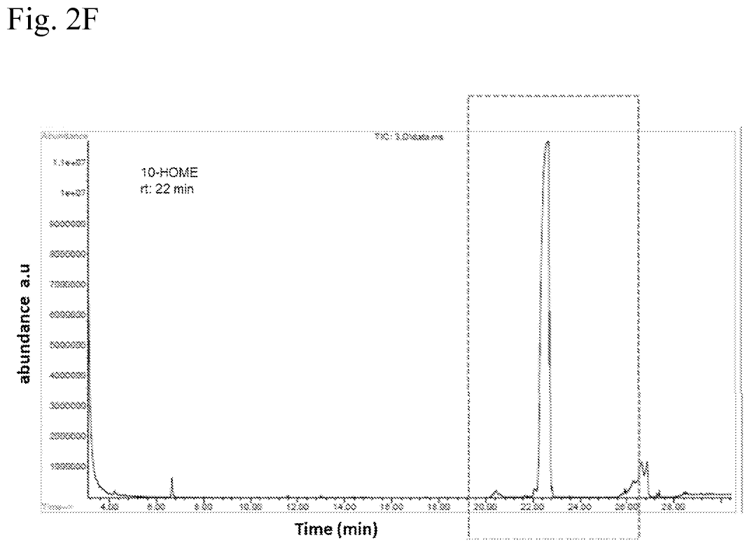

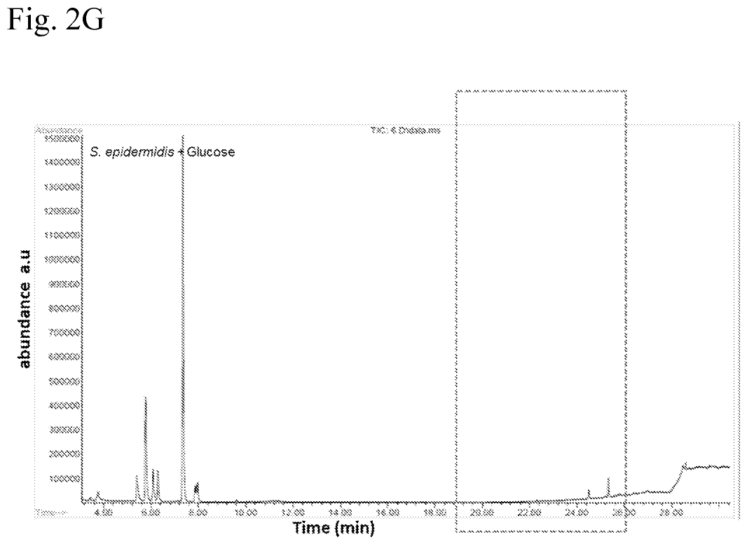

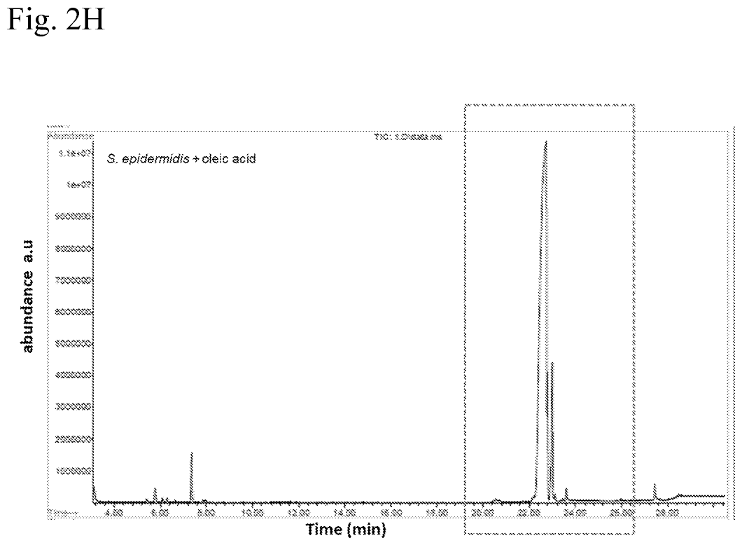

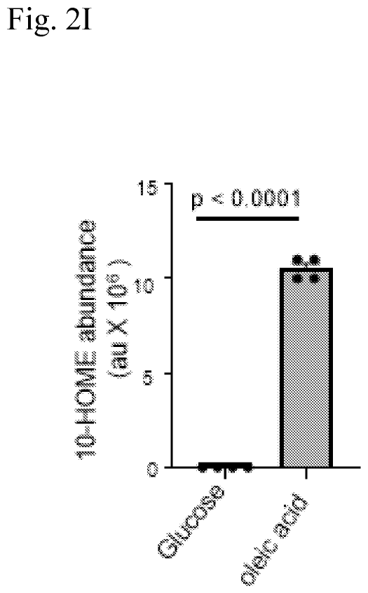

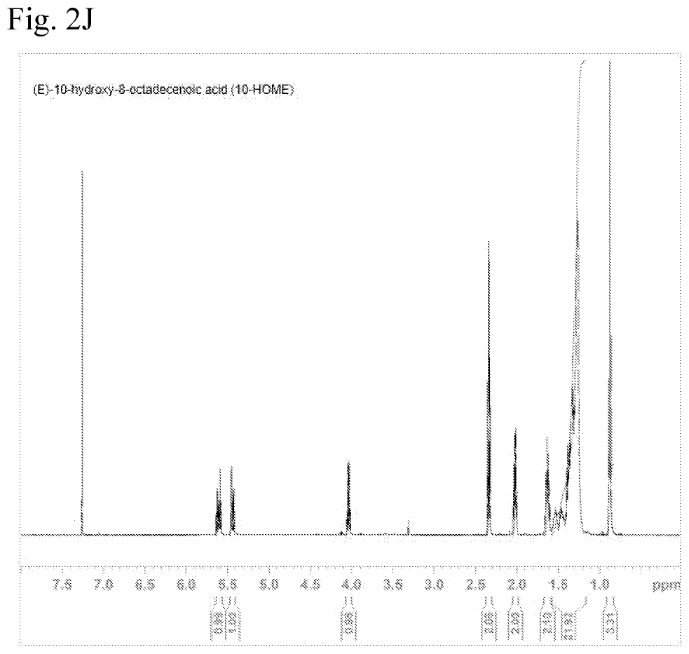

The oxylipin 10-hydroxy-(8E)-octadecenoic acid (10-HOME) is formed by the oxidation of oleic acid (FIG. 2A). The oxylipin 10-HOME has been reported to inhibit flagellum-driven swimming and swarming motilities and stimulate the formation of bacterial biofilms in vitro. Elevated levels of 10-HOME were observed through mass spectrometry in implant associated samples of BII compared to non-BII samples (FIG. 2B). Positive correlation was observed between bacterial abundance and concentration of 10-HOME (FIG. 2D). Formation of 10-HOME was detected when biofilm forming S. epidermidis was cultured in vitro with oleic acid as source of carbon. The oxylipin 10-HOME was synthesized in the laboratory in light isotope and heavy isotope forms to be used as analytical standards and for biological testing. The synthesized 10-HOME was validated through thin layer chromatography and NMR spectroscopy.

1. A process for forming a compound of the formula I 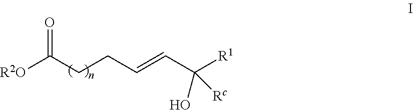
or a salt thereof, wherein
R¹ is C₁-C₁₀ alkyl or C₁-C₁₀ alkyl having one or more of the hydrogen atoms in C₁-C₁₀ alkyl being independently, substituted by deuterium;
R² is H, C₁-C₆ alkyl, C₆-C₁₀ aryl, heterocycloalkyl, cycloalkyl, or heteroaryl, optionally wherein one or more hydrogen atoms in C₁-C₆ alkyl, C₆-C₁₀ aryl, heterocycloalkyl, cycloalkyl, or heteroaryl is independently substituted by deuterium;
R^{c} is H or D; and
n is an integer from 1-7; the process comprising the following procedures:
I)
(a) contacting a compound of formula II 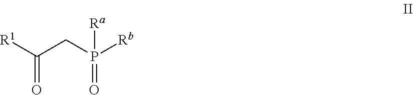
wherein R¹ is as defined above, and each of R^{a} and R^{b} is independently —OC₁-C₆ alkyl, —Oaryl, —Oheteroaryl; with a compound of formula III 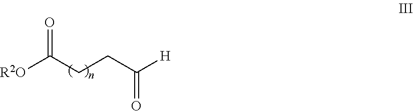
wherein R² and n are as defined above, in the presence of a base to form a compound of formula IV 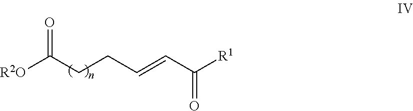
wherein R¹ and R² are as defined above; and
(b) contacting a compound of formula IV with a reducing agent to form the compound of formula I 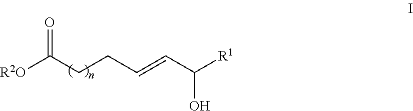
wherein R¹ and R² are as defined above; and optionally hydrolyzing the compound of formula I wherein R² is not H to form a compound of formula I wherein R² is H; or
II)
(c) contacting a compound of formula V
PG-O—C₁-C₉ alkyl-LG V
wherein PG is a hydroxy protecting group and LG is a leaving group, and each hydrogen atom in C₁-C₉ alkyl is optionally substituted by deuterium, with a compound of formula R³-MX, wherein R³ is C₁-C₉ alkyl, wherein each hydrogen atom in C₁-C⁹ alkyl is optionally substituted by deuterium, M is a metal, and X is a halogen, to form a compound of formula VI
PG-O—CH₂R¹ VI
wherein R¹ is as defined above and each hydrogen atom in —CH₂R¹ is optionally substituted by deuterium; or
(d) deprotecting a compound of formula VI into a compound of formula VII
HO—CH²R¹ VII
wherein each hydrogen atom in —CH²R¹ is optionally substituted by deuterium;
(e) oxidizing a compound of formula VII to form a compound of formula VIII
R^{D}O(O)C—R¹ VIII
wherein R^{D} is H or C₁-C₆ alkyl; and
(f) contacting a compound of formula VIII with (Rₐ)(R_{b})(CH₃)P(O) in the presence of a base to form a compound of formula II; and
(g) oxidatively cleaving a C₃-C₁₀ cycloalkene with a reducing agent to produce a linearized dicarbonyl and optionally esterifying the product to form a compound of formula III which can be used to generate the compound of /Formula I using steps (a) and (b).

2. The process of claim 1 comprising step (a) and step (b).

3. The process of claim 2 further comprising steps (c)-(g).

4. The process of claim 1, comprising steps (c)-(g).

5. The process of claim 1 wherein
R^{c} is H; and
R¹ is C₂-C₁₀ alkyl, or C₂-C₁₀ alkyl where one or more hydrogen atoms in C₂-C₁₀ alkyl is independently, substituted by deuterium.

6. The process of claim 5, wherein R¹ is C₆-C₁₀ alkyl, or C₆-C₁₀ alkyl where one or more hydrogen atoms in C₆-C₁₀ alkyl is independently, substituted by deuterium

7. The process of claim 6, wherein R¹ comprises at least three deuteriums.

8. The process of claim 1, wherein the compound of formula I is 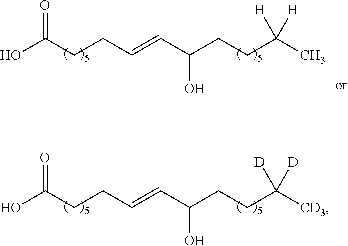
or a salt thereof.

9. A compound of formula I 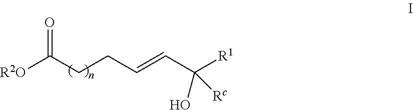
wherein
R¹ is C₁-C₁₀ alkyl and at least one hydrogen atom in C₁-C₁₀ alkyl is substituted by deuterium;
R² is H, C₁-C₆ alkyl, C₆-C₁₀ aryl, heterocycloalkyl, cycloalkyl, or heteroaryl, wherein each hydrogen atom in C₁-C₆ alkyl, C₆-C₁₀ aryl, or 3-7 membered heteroaryl is optionally substituted by deuterium;
R^{c} is H or D; and
n is an integer from 1-7;
or a salt thereof.

10. The compound or salt of claim 9, wherein R² is H.

11. The compound or salt of claim 10, wherein R^{C} is H.

12. The compound or salt of claim 11, wherein R¹ includes at least three deuteriums.

13. The compound or salt of claim 12, wherein the compound is

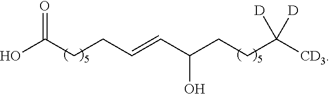

14. A method for detecting a biofilm in a patient, the method comprising
collecting a sample from a patient;
adding to the sample a compound or salt thereof of claim 9, to form a spiked sample; and
analyzing the spiked sample with mass spectrometry.

15. The method of claim 14, wherein said compound has the general formula of 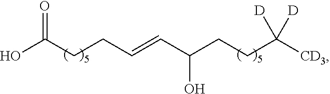
or a salt thereof.

16. The method of claim 14, wherein the sample is from a breast implant.

17. A method for detecting a biofilm in a patient, the method comprising
collecting a sample from a patient;
adding to the sample a compound or salt thereof made according to the process of claim 1, to form a spiked sample; and
analyzing the spiked sample with mass spectrometry.

18. The method of claim 17, wherein said compound has the general formula of 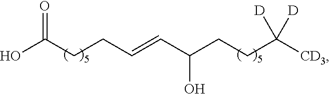
or a salt thereof.

19. The method of claim 18, wherein the sample is from a breast implant.
